(19) United States
(12) Patent Application Publication
(10) Pub. No.: US 2026/0026969 A1
(43) Pub. Date: Jan. 29, 2026

(54) OPHTHALMIC SURGICAL INSTRUMENTS

(71) Applicant: Alcon Inc., Fribourg (CH)

(72) Inventor: Michael Sam Cardamone, Keller, TX (US)

(21) Appl. No.: 19/259,003

(22) Filed: Jul. 3, 2025

Related U.S. Application Data
(60) Provisional application No. 63/674,439, filed on Jul. 23, 2024.

(51) Int. Cl. A61F 9/007; A61B 17/00

(52) U.S. Cl. CPC A61F 9/00736; A61B 2017/00199; A61B 2017/00238; A61B 2217/005

(57) ABSTRACT

An ophthalmic surgical instrument includes a handpiece configured to be held by a user, and a working tip coupled to a distal end of the handpiece that includes a proximal opening configured to be disposed outside an eye, a distal opening configured to be disposed inside the eye and expose a tool to the inside of the eye, and a channel between the openings. When the distal opening is disposed inside the eye and the proximal opening is uncovered the proximal opening permits the channel to be in fluid communication with ambient atmosphere, the channel is configured to enable passive aspiration of fluid from inside the eye, such that the fluid flows from the distal to the proximal opening and into the ambient atmosphere. The tool comprises forceps, scissors, a delamination spatula, a pick, an illumination fiber, or a laser fiber.

### INTRODUCTION

Vitreoretinal procedures frequently require multiple ophthalmic surgical instruments. For example, certain vitreoretinal procedures may require one ophthalmic surgical instrument for operating and/or irrigating fluid inside an eye, and another ophthalmic surgical instrument for aspirating fluid out from within the eye. A surgeon may therefore need to switch between the different ophthalmic surgical instruments one or more times throughout the procedure to maintain stability of the eye's intraocular pressure (IOP) and anterior chamber (AC).

However, inserting and removing different ophthalmic surgical instruments throughout the course of a procedure can result in unwanted and unintentional trauma to ocular tissues, which can potentially lead to other ophthalmic complications. Further, having to switch between different ophthalmic surgical instruments throughout the procedure may increase the amount of time needed to complete the procedure, which further increases patient risk.

### BRIEF SUMMARY

The present disclosure relates generally to ophthalmic surgical instruments for performing ophthalmic surgical procedures, such as vitrectomy, retinal detachment repair, macular hole surgery, retinal laser surgery, etc.

In certain embodiments, an ophthalmic surgical instrument is provided. The ophthalmic surgical instrument includes a handpiece configured to be held by a user, and a working tip coupled to a distal end of the handpiece. The working tip comprises a proximal opening configured to be disposed outside an eye, a distal opening configured to be disposed inside the eye and expose a tool to the inside of the eye, and a channel between the distal opening and the proximal opening. When the distal opening of the working tip is disposed inside the eye and the proximal opening is uncovered: (1) the proximal opening permits the channel to be in fluid communication with ambient atmosphere, and (2) the channel is configured to enable passive aspiration of fluid from inside the eye, such that the fluid flows from the distal opening to the proximal opening and into the ambient atmosphere. The tool comprises one of forceps, scissors, a delamination spatula, a pick, an illumination fiber, or a laser fiber.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1 illustrates a cross-sectional side view of an eye with a side view of an example working tip of an ophthalmic surgical instrument inserted therein, according to certain embodiments.

FIG. 2A is an isometric view of the example working tip shown in FIG. 1, according to certain embodiments.

FIG. 2B is a cross-sectional view of the example working tip shown in FIG. 2A, according to certain embodiments.

FIGS. 3A-3B are cross-sectional views of another example working tip, according to certain embodiments.

FIG. 4 is a cross-sectional view of an example ophthalmic surgical instrument including the working tip shown in FIGS. 3A-3B, according to certain embodiments.

FIGS. 5A-5B are isometric views showing example tools for the working tips shown in FIGS. 1, 2A-2B, 3A-3B, and 4, according to certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

It will be readily understood that the components of the embodiments as generally described herein and illustrated in the appended Figures can be arranged and designed in a wide variety of different configurations. Thus, the following more detailed description of various embodiments, as represented in the Figures, is not intended to limit the scope of the present disclosure but is merely representative of various embodiments. While the various aspects of the embodiments are presented in the Figures, the Figures are not necessarily drawn to scale unless specifically indicated.

Reference throughout this specification to the term “distal” refers to a system, device, component, end, portion, or segment that is disposed closer to a patient and/or further from a console during an ophthalmic procedure; and the term “proximal” refers to the system, device, component, end, portion, or segment that is disposed further from the patient and/or closer to the console during the ophthalmic procedure.

During vitreoretinal surgeries (e.g., vitrectomy, retinal detachment repair, macular hole surgery, retinal laser surgery, epiretinal membrane (ERM) peel, etc.), fluid (e.g., a balanced salt solution (BSS) or other irrigation solution) may be irrigated into the eye to maintain stability of the eye's intraocular pressure (IOP) and anterior chamber (AC). In turn, the fluid within the eye may also need to be aspirated out of the eye to prevent an excess increase in the eye's IOP, which can result in unwanted and unintentional trauma to ocular tissues, leading to potentially permanent damage to the eye, disturbing its function and/or causing various other complications.

Current vitreoretinal surgeries often employ an ophthalmic surgical instrument, such as an aspiration probe, that is used to aspirate fluid out of the eye along with other ophthalmic surgical instruments for performing various surgical functionalities, such as forceps, a finesse loop, an illumination fiber, a laser fiber, a delamination spatula, a pick, scissors, etc. As such, a surgeon may need to switch between the different ophthalmic surgical instruments one or more times throughout the surgery, as the surgeon can only hold two instruments, without the assistance of a surgical assistant. However, exchanging different instruments into and out of the eye throughout the surgery increases the likelihood of unwanted and unintentional trauma to ocular tissues, and may also increase a duration of the surgery, which further increases risk to the patient.

Accordingly, the embodiments described herein provide ophthalmic surgical instruments that provide a channel for passive aspiration, thereby allowing fluid to flow out of the eye. For example, each of the ophthalmic surgical instruments herein are multifunctional instruments, such that they each perform a certain surgical functionality (e.g., forceps, finesse loop, a laser fiber, an illumination fiber, a delamination spatula, a pick, scissors, etc.) while also acting as a passive aspiration instrument. The multifunctional instruments described herein, therefore, reduce the need to switch between different instruments throughout the surgery, the likelihood of unwanted and unintentional trauma to ocular tissues, and the duration of the surgery.

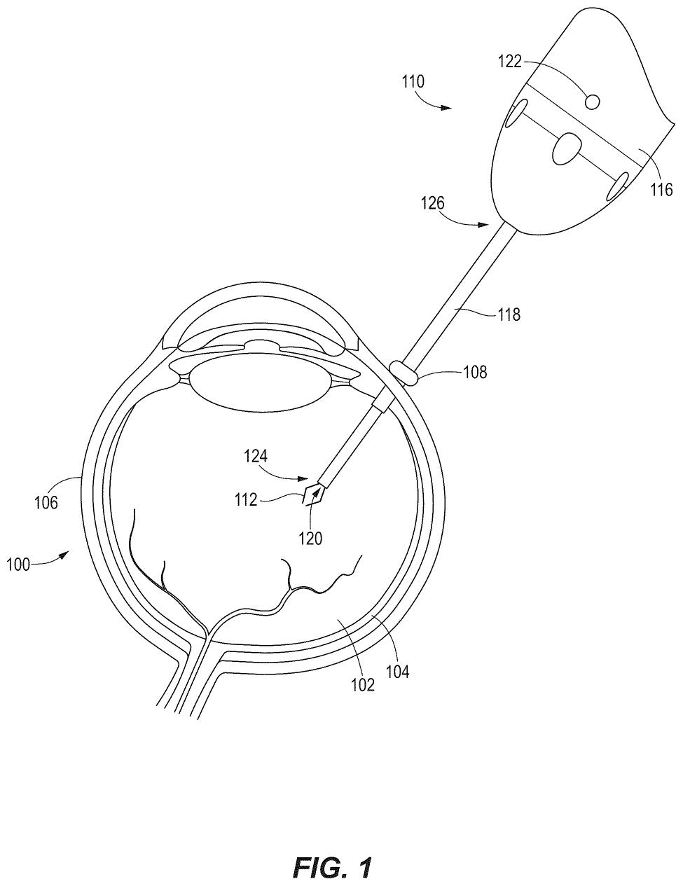

FIG. 1 illustrates a cross-sectional side view of an eye 100 with an example working tip 110 of an ophthalmic surgical instrument inserted therein, according to certain embodiments. In FIG. 1, the eye 100 includes vitreous 102, a retina 104, and a sclera 106. The working tip 110 is inserted into the eye 100 through a trocar cannula 108 that is inserted into an incision in the sclera 106, as would be understood by skilled persons. The working tip 110 includes an adapter 116, a cannula 118 coupled to the adapter 116, and a tool 112 extending from the cannula 118.

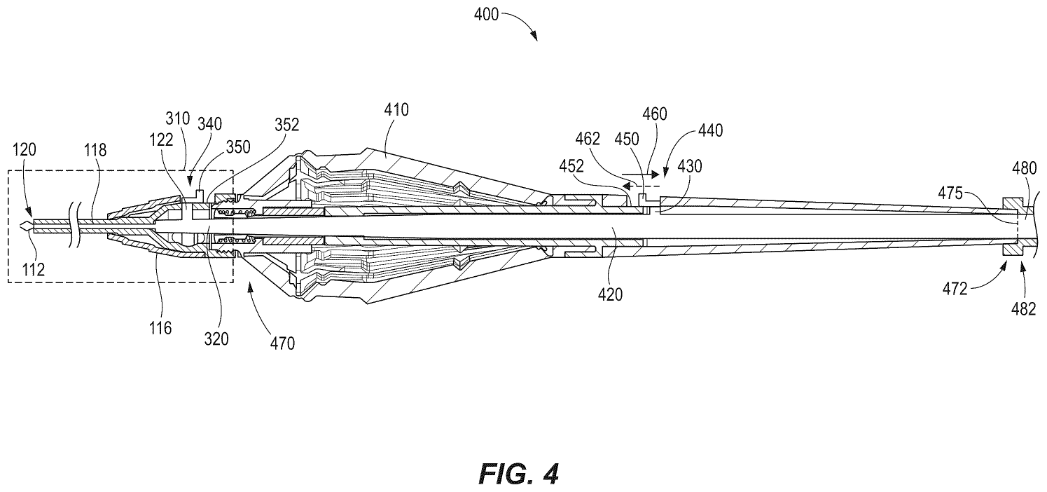

The tool 112 is configured to perform a certain surgical functionality within the eye 100. In FIG. 1, the tool 112 is forceps; however, the tool 112 may instead be any other tool used in vitreoretinal surgeries, such as a finesse loop (e.g., as shown in FIG. 4B), a laser fiber, an illumination fiber, a delamination spatula, a pick, scissors, etc.

In certain embodiments, the adapter 116 is configured to be coupled to a distal end of a handpiece that can be held by the user (e.g., see FIG. 4) and control functionality of the tool 112. The adapter 116 includes a proximal opening 122 configured to be disposed outside the eye 100. The proximal opening 122 is shown as being circular, but may also be rectangular, elliptical, or any other similar shape.

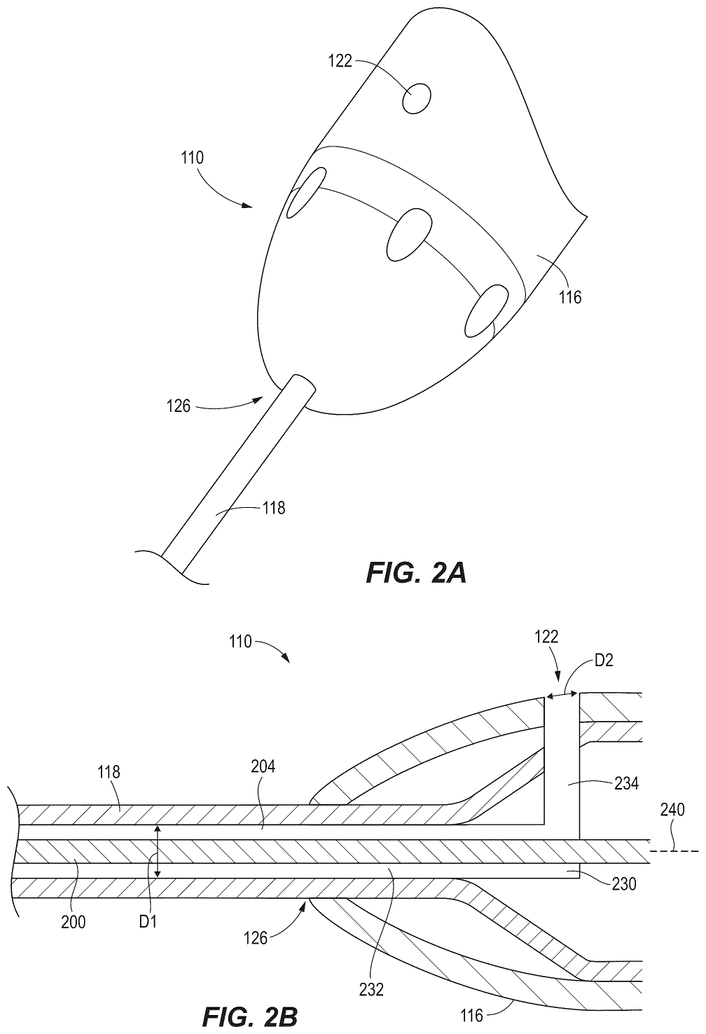

The cannula 118 is coupled to the adapter 116 at its proximal end 126 and extends distally from the adapter 116. The cannula 118 includes a distal opening 120 at a distal end 124, the distal opening 120 configured to be disposed inside the eye 100 and expose the tool 112 to the inside of the eye 100. The cannula 118 also defines a channel (e.g., channel 204 shown in FIG. 2B) between the distal opening 120 and the proximal opening 122.

During use of the working tip 110, for example, as shown in FIG. 1, the distal opening 120 of the cannula 118 is disposed inside the eye 100, and the proximal opening 122 of the adapter 116 is disposed outside the eye 100. The proximal opening 122 thereby permits the channel to be in fluid communication with ambient atmosphere outside the eye 100 when the distal opening 120 is disposed inside the eye 100. In other words, an airflow and/or fluidic pathway between inside the eye 100 and the ambient atmosphere outside the eye 100 is enabled by the distal opening 120 being disposed inside the eye 100 and the proximal opening 122 being disposed outside the eye 100.

Thus, when the distal opening 120 is disposed inside the eye 100 and the proximal opening 122 is uncovered, a pressure difference (or a pressure gradient force) can be created between the openings 120, 122 due to the pressure inside the eye 100 being greater than the pressure outside the eye 100. This pressure difference causes fluid within the eye 100 to flow into the distal opening 120, through the channel in the cannula 118, and out of the proximal opening 122 into the ambient atmosphere, which is herein referred to as passive aspiration. When the pressure inside the eye 100 increases, for example, due to fluid that is irrigated into the eye 100 during an ophthalmic procedure, a rate at which the fluid is passively aspirated may increase.

This flow of the fluid through the working tip 110 can be controlled by the user. For example, the user can control the flow of the fluid by covering the proximal opening 122 of the adapter 116 with a finger. When the distal opening 120 of the working tip 110 is disposed inside the eye 100, covering the proximal opening 122 (e.g., with a finger) eliminates the pressure difference between the inside of the eye 100 and the channel in the working tip 110, which stops the fluid from exiting through the proximal opening 122. When the user removes their finger from covering the proximal opening 122, the pressure difference can be reestablished, and the fluid within the eye 100 may continue (or resume) flowing out of the proximal opening 122.

In certain embodiments, the fluid flowing out of the proximal opening 122 may ultimately be collected by a drape bag disposed around or near an exterior portion of the eye 100 during the ophthalmic procedure. As an example, the fluid may flow directly out of the proximal opening 122, onto an exterior portion of the eye 100, and into the drape bag. As another example, the fluid may flow out of the proximal opening 122, onto an exterior portion of the working tip 110 and/or exterior portion of the eye 100, then into the drape bag. In certain embodiments, the fluid collected by the drape bag may drain into a drainage bag, which helps maintain a sterile area in and around the eye 100.

FIG. 2A is an isometric view of the example working tip 110 shown in FIG. 1, according to certain embodiments. FIG. 2B is a cross-sectional view of the example working tip 110 shown in FIG. 2A, according to certain embodiments. Accordingly, FIGS. 2A-2B are described together herein for clarity.

In addition to the tool 112, the adapter 116, and the cannula 118 shown in FIG. 1, the working tip 110 shown in FIG. 2A further includes a rod 200 that is coupled to the tool 112, and which extends distally through and/or from the adapter 116. In certain embodiments, the rod 200 is operably coupled to the handpiece to which the adapter 116 is coupled. The cannula 118 is disposed over at least a portion of the rod 200 and defines a channel 204 therebetween.

The cannula 118 has a diameter (D1) that is, for example, between 0.05 millimeter (mm) and 5 mm (e.g., between 0.1 mm and 4.95 mm, 0.15 mm and 4.90 mm, 0.2 mm and 4.85 mm, or 0.25 mm and 4.8 mm). Additionally, the proximal opening 122 has a diameter (D2) that is, for example, between 0.05 mm and 20 mm (e.g., between 0.1 mm and 19.5 mm, 0.15 mm and 19 mm, 0.2 mm and 18.5 mm, or 0.25 mm and 18 mm). In certain embodiments, the diameter (D2) of the proximal opening 122 is equal to or greater than the diameter (D1) of the cannula 118. In certain embodiments, sizing of the diameters D1 and D2 corresponds to a gauge of an ophthalmic surgical instrument.

The channel 204 defined by the cannula 118 is disposed between the distal opening 120 and the proximal opening 122, extends at least partially into the adapter 116, and is configured to enable passive aspiration of fluid inside the eye 100, such that the fluid flows from the distal opening 120 to the proximal opening 122 and into an ambient atmosphere as discussed with reference to FIG. 1. Within the adapter 116, the channel 204 includes a first portion 232 that is parallel to a longitudinal axis 240 of the working tip 110, and a second portion 234 that is angled (i.e., not parallel) in relation to the longitudinal axis 240. The second portion 234 fluidly couples first portion 232 to the proximal opening 122.

The first portion 232 and the second portion 234 define an angled portion 230 therebetween. The angled portion 230 is defined by an angle created by the second portion 234 relative to the first portion 232. The angled portion 230 is angled at approximately 90° (degrees) (e.g., 90°±15°). However, in certain other embodiments, angled portion 230 can be angled differently, such as between 5° to 175°. Further, in certain embodiments, in addition to angled portion 230, the working tip 110 may include one or more other angled portions with the angle(s) between 5° and 175°, 15° and 165°, 25° and 155°, or 35° and 145°.

The working tip 110 may be used in a manner similar to the working tip 110 such that during use of the working tip 110, the distal opening 120 of the cannula 118 is disposed inside the eye 100, and the proximal opening 122 of the adapter 116 is disposed outside the eye 100. The pressure gradient force created by the difference in pressures inside and outside the eye 100 then causes fluid within the eye 100 to flow into the distal opening 120, through the channel 204, and out of the proximal opening 122. The user can further control the flow of the fluid by covering or uncovering the proximal opening 122 (e.g., with a finger).

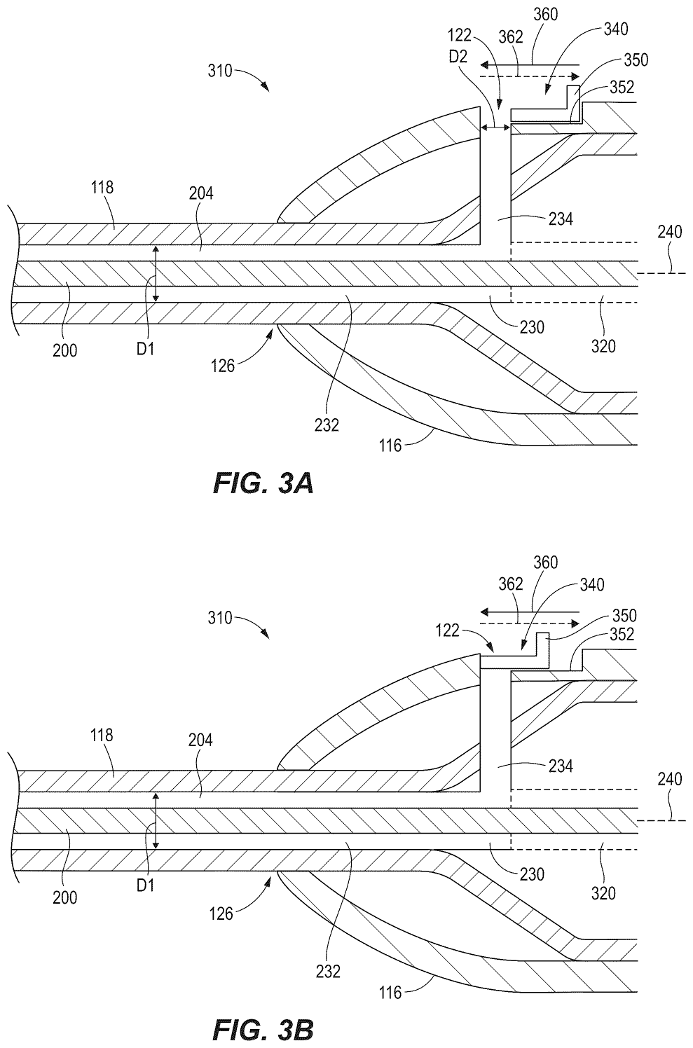

FIGS. 3A-3B are cross-sectional views of another example working tip 310, according to certain embodiments. Accordingly, FIGS. 3A-3B are described together herein for clarity.

The working tip 310 shown in FIGS. 3A-3B is similar to the working tip 110 shown in FIGS. 1 and 2A-2B, but further includes a valve mechanism 340 configured to control flow of the fluid out of the proximal opening 122 and into the ambient atmosphere by covering the proximal opening 122. The valve mechanism 340 includes a sliding cover 350 that is moveably disposed in a housing 352 of the adapter 116. The sliding cover 350 may be movingly coupled to the housing 352 by, for example, a sliding pin or groove, which allows sliding cover 350 to slide over the proximal opening 122.

As illustrated in FIG. 3A, when the sliding cover 350 is in a first position, the sliding cover 350 does not cover the proximal opening 122 and fluid can flow through and out of the proximal opening 122. In the first position, the sliding cover 350 does not entirely block the proximal opening 122, and the pressure difference between the distal opening 120 and the proximal opening 122 is maintained, thereby causing the flow of the fluid into the distal opening 120, through the channel 204, and out of the proximal opening 122. Note that, friction and/or stoppers between the sliding cover 350 and the housing 352 can be used to keep the sliding cover 350 in place (e.g., the first position) until otherwise moved by the user (e.g., a surgeon).

The user can place the sliding cover 350 in a second position by applying force to the sliding cover 350, thereby moving it distally (in a first direction 360) to fully block or cover the proximal opening 122. When the sliding cover 350 is in the second position, the pressure difference between the distal opening 120 and the proximal opening 122 is eliminated (or is not established), thereby stopping (or pausing) the flow of the fluid into the distal opening 120, through the channel 204, and out of the proximal opening 122. Similarly, once placed in the second position, friction and/or stoppers between the sliding cover 350 and the housing 352 can cause the sliding cover 350 to remain in the second position until moved by the user.

After placing sliding cover 350 in the second position, shown in FIG. 3B, the user may return the sliding cover 350 to the first position shown in FIG. 3A by moving the sliding cover 350 proximally (in a second direction 362). When the sliding cover 350 is no longer covering the proximal opening 122, the fluid within the eye 100 may continue (or resume) flowing through the channel 204 and out of the proximal opening 122.

Thus, the sliding cover 350 is configured to transition (or move) between the first position and second position shown in FIGS. 3A and 3B, respectively, which allows the user to control the flow of the fluid through the channel 204. Further, because the sliding cover 350 is configured to cover the proximal opening 122 in the second position shown by FIG. 3B, using the working tip 310 with the valve mechanism 340 eliminates the need for the user to cover the proximal opening 122 with their finger, for example, as described with reference to working tip 110.

In some embodiments, the sliding cover 350 may be configured to move relative to the housing 352 by interacting with a guiding mechanism. As an example, the guiding mechanism may be rails, grooves, tracks, etc., which correspond with a shape of the sliding cover 350, keep the sliding cover 350 aligned with the proximal opening 122, and/or prevent the sliding cover 350 from rotating, tilting, or falling out while it moves between the first and second positions. Further, in some embodiments, lubricant, bearings, and/or rollers may be implemented with the guiding mechanism to facilitate smooth movement between the sliding cover 350 and the housing 352.

In some embodiments, a spring mechanism is configured to keep the sliding cover 350 in the second position shown in FIG. 3B. For example, a spring in the housing 352 is configured to decompress and engage with the sliding cover to push it distally to fully block or cover the proximal opening 122. When the user moves the sliding cover 350 proximally back to the first position shown in FIG. 3A, the spring compresses and the sliding cover 350 uncovers the proximal opening 122. As such, the user may need to keep their finger on the sliding cover 350 to prevent the spring from returning the sliding cover 350 to the second position. In some other embodiments, a similar spring mechanism can instead be configured to keep the sliding cover 350 in the first position shown in FIG. 3A. In such embodiments, the user may need to keep their finger on the sliding cover 350 to prevent the spring from returning the sliding cover 350 to the first position.

Note that although the valve mechanism 340 is shown as including the sliding cover 350, other types of valve mechanisms are within the scope of this disclosure. For example, a valve mechanism that instead includes a latch, a plug, a screw, or other similar component configured to cover the proximal opening 122 may be used. For example, in certain embodiments, a latch may be coupled to the housing 352 and can be configured to flip (e.g., via a hinge or other similar coupling mechanism) between an open first state where the latch does not cover the proximal opening 122 (similar to as shown in FIG. 3A), and a closed second state where the latch covers the proximal opening 122 (similar to as shown in FIG. 3B). In another example, a plug or screw may be inserted in the proximal opening 122 (e.g., via slots or threads in the proximal opening 122) to cover the proximal opening 122 (similar to as shown in FIG. 3B) and may be removed from the proximal opening 122 so that the plug or screw does not cover the proximal opening 122 (similar to as shown in FIG. 3A).

In certain embodiments, as shown by the dashed lines in FIGS. 3A-3B, the channel 204 may optionally extend past the second portion 234. For example, the channel 204 may optionally include an extended portion 320 that is configured to fluidly couple to a channel in the corresponding handpiece, which may include an additional or alternative opening and valve mechanism similar to the proximal opening 122 and the valve mechanism 340 of the working tip 310. In other words, in certain embodiments, an ophthalmic surgical instrument may include an opening and valve mechanism at the working tip and/or at the handpiece. An example of an ophthalmic surgical instrument which includes an opening and valve mechanism at the working tip and the handpiece is described in further detail with reference to FIG. 4.

FIG. 4 is a cross-sectional view of an example ophthalmic surgical instrument 400 including the working tip 310 shown in FIGS. 3A-3B, according to certain embodiments. The ophthalmic surgical instrument 400 includes a handpiece 410 including a channel 420 (referred to herein as a “handpiece channel”), an opening 430 (referred to herein as a “handpiece opening”), and a valve mechanism 440 (referred to herein as a “handpiece valve mechanism”). The handpiece channel 420 couples to the extended portion 320 of the channel 204 at a distal end 470 of the handpiece 410, and to an aspiration tube (or line) 480 via an aperture 475 at a proximal end 472 of the handpiece 410. That is, a distal end 482 of the aspiration tube 480 couples to the aperture 475 of the handpiece 410 at the proximal end 472 of the handpiece 410.

The handpiece valve mechanism 440 is similar to the valve mechanism 340 described with reference to FIGS. 3A-3B but is arranged in an orientation opposite to that of the valve mechanism 340. For example, the handpiece valve mechanism 440 includes a handpiece sliding cover 450 that is moveably disposed in a handpiece housing 452, the handpiece sliding cover 450 configured to move proximally in a first direction 460 and distally in a second direction 462.

As illustrated in FIG. 4, the handpiece sliding cover 450 has been moved from an open, first position (similar to the first position of the sliding cover 350 shown in FIG. 3A), to a closed, second position (similar to the second position of the sliding cover 350 shown in FIG. 3B). A user (e.g., a surgeon) may move the handpiece sliding cover 450 from the first position to the second position by moving the handpiece sliding cover 450 proximally in the first direction 460.

In the second position illustrated by FIG. 4, the handpiece sliding cover 450 is covering the handpiece opening 430. For example, in the second position, the handpiece sliding cover 450 has been completely moved to cover the handpiece opening 430. After the handpiece sliding cover 450 has reached the second position shown in FIG. 4, the user may return the handpiece sliding cover 450 to the first position by moving the handpiece sliding cover 450 distally in the second direction 462. Thus, the handpiece sliding cover 450 is configured to transition (or move) between the first position and second position.

During use of the ophthalmic surgical instrument 400, when both the valve mechanism 340 and the handpiece valve mechanism 440 cover the proximal opening 122 and the handpiece opening 430, respectively, the pressure difference between the distal opening 120 and the proximal opening 122 or the handpiece opening 430 is eliminated (or cannot be created). Eliminating the pressure difference thereby stops (or pauses) the flow of the fluid into the distal opening 120, through the channels 204, 420, and out of the openings 122 or 430. As such, passive aspiration of the fluid does not occur.

Instead, fluid within the eye 100 can be actively aspirated into the distal opening 120 and through the channels 204, 420 via the aspiration tube 480, which may be connected to a surgical console at its proximal end. For example, the aspiration tube 480 may be proximally coupled to an aspiration source in the surgical console, whereby the aspiration source is configured to provide active aspiration of the fluid through the ophthalmic surgical instrument 400 when both openings 122, 430 are covered. In certain embodiments, the user can control a flow rate of the fluid using the surgical console and/or other devices connected thereto.

However, when passive aspiration of the fluid within the eye 100 is desired, the active aspiration provided by the surgical console is stopped (e.g., turned off) and one of the openings 122 or 430 are uncovered. For example, one of the sliding covers 350 or 450 is moved to the open, first position, which thereby establishes the pressure difference. The pressure difference causes the fluid to flow into the distal opening 120, through the channels 204, 420, and out one of the openings 122 or 430.

As an example, when the handpiece sliding cover 450 is in the closed, second position, and the sliding cover 350 is in the open, first position, the fluid is configured to flow into the distal opening 120, through the channel 204, and out of the proximal opening 122. As another example, when the sliding cover 350 is in the closed, second position, and the handpiece sliding cover 450 is in the open, first position, the fluid is configured to flow into the distal opening 120, through the channels 204, 420, and out of the handpiece opening 430.

Further, although the ophthalmic surgical instrument 400 is shown as including two valve mechanisms 340, 440 and openings 122, 430, the ophthalmic surgical instrument 400 may only include one of the two valve mechanisms 340, 440 and openings 122, 430. Alternatively, the ophthalmic surgical instrument 400 may also include more than two valve mechanisms and more than two openings.

By allowing the fluid within the eye 100 to flow out through the working tips 110, 310 via passive aspiration, or out through the ophthalmic surgical instrument 400 via active aspiration, the IOP and AC stability of the eye 100 can be maintained with fewer exchanges of different instruments in the eye 100. Thus, reducing the exchange of different instruments allows surgeries to be more efficient and lessens risks for patients.

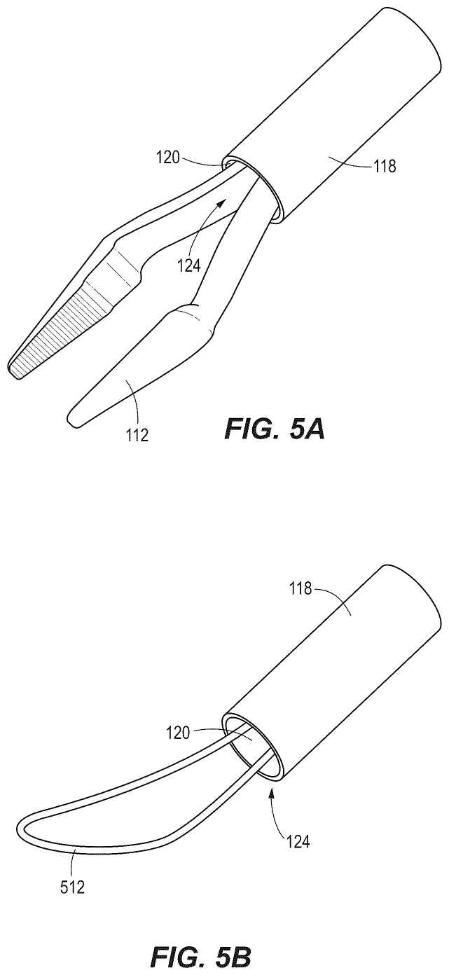

FIGS. 5A-5B are isometric views showing example tools for the working tips 110, 310 shown in FIGS. 1, 2A-2B, 3A-3B, and 4 according to certain embodiments.

In FIG. 5A, tool 112 is forceps, which extend from the distal end 124 of the cannula 118 through the distal opening 120. As known to one of ordinary skill in the art, the forceps can be activated by a mechanism that either retracts and protracts the forceps or alternatively retracts and protracts the cannula 118. In FIG. 5B, tool 512 is a finesse loop, which extends from the distal end 124 of the cannula 118 through the distal opening 120. Similarly, as known to one of ordinary skill in the art, the finesse loop can be activated by a mechanism that either retracts and protracts the finesse loop or alternatively retracts and protracts the cannula 118.

Note that either of the tools 112 or 512 can be implemented with either of the working tips 110 or 310 shown in FIGS. 1, 2A-2B, 3A-3B, and 4. Further, in certain embodiments, other tools may also be implemented with either of the working tips 110 or 310. For example, the working tips 110 or 310 may include tools of illumination fibers, laser fibers, delamination spatulas (and illuminated delamination spatulas), picks (and illuminated picks), scissors, etc. For an illumination fiber and/or a laser fiber to achieve passive aspiration, the probe and/or the fiber may be at least partially disposed within a cannula and an adapter, for example, similar to the cannula 118 and the adapter 116 disposed over the rod 200.

The present disclosure may be embodied in other specific forms without departing from its spirit or essential characteristics. The described embodiments are to be considered in all respects only as illustrative and not restrictive. The scope of the present disclosure is, therefore, indicated by the appended Claims rather than by this Detailed Description. All changes which come within the meaning and range of equivalency of the Claims are to be embraced within their scope.

Reference throughout this specification to features, advantages, or similar language does not imply that all the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Thus, discussions of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize, in light of the description herein, that the disclosure can be practiced without one or more of the specific features or advantages of a particular embodiment. In other instances, additional features and advantages may be recognized in certain embodiments that may not be present in all embodiments of the present disclosure.

Reference throughout this specification to “one embodiment”, “an embodiment”, or similar language means that a particular feature, structure, or characteristic described in connection with the indicated embodiment is included in at least one embodiment of the present disclosure. Thus, the phrases “in one embodiment”, “in an embodiment”, and similar language throughout this specification may, but do not necessarily, all refer to the same embodiment.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein but are to be accorded the full scope consistent with the language of the claims.

What is claimed is:

1. An ophthalmic surgical instrument, the ophthalmic surgical instrument comprising:
a handpiece configured to be held by a user; and
a working tip coupled to a distal end of the handpiece, the working tip comprising:
a proximal opening configured to be disposed outside an eye;
a distal opening configured to be disposed inside the eye and expose a tool to the inside of the eye; and
a channel between the distal opening and the proximal opening, wherein when the distal opening of the working tip is disposed inside the eye and the proximal opening is uncovered:
the proximal opening permits the channel to be in fluid communication with ambient atmosphere, and
the channel is configured to enable passive aspiration of fluid from inside the eye, such that the fluid flows from the distal opening to the proximal opening and into the ambient atmosphere; and
the tool comprising one of forceps, scissors, a delamination spatula, a pick, an illumination fiber, or a laser fiber.

2. The ophthalmic surgical instrument of claim 1, wherein the working tip further comprises:
an adapter configured to couple the working tip to the handpiece, wherein:
the proximal opening is disposed through the adapter;
a rod extending distally from the adapter; and
a cannula disposed over a portion of the rod, wherein:
the distal opening is disposed at a distal end of the cannula.

3. The ophthalmic surgical instrument of claim 2, wherein the channel comprises:
a first portion that is parallel to a longitudinal axis of the working tip; and
a second portion that is not parallel in relation to the longitudinal axis of the working tip,
wherein the second portion fluidly couples the first portion to the proximal opening.

4. The ophthalmic surgical instrument of claim 2, wherein the cannula extends partially into the adapter.

5. The ophthalmic surgical instrument of claim 1, wherein when the distal opening of the working tip is disposed inside the eye, covering the proximal opening eliminates a pressure difference between the inside of the eye and inside of the channel.

6. The ophthalmic surgical instrument of claim 1, further comprising a valve mechanism configured to control flow of the fluid out of the proximal opening and into the ambient atmosphere.

7. The ophthalmic surgical instrument of claim 6, wherein the valve mechanism comprises a sliding cover configured to cover the proximal opening.

8. The ophthalmic surgical instrument of claim 1, wherein a diameter of the proximal opening is equal to or greater than a diameter of the channel.

9. The ophthalmic surgical instrument of claim 1, wherein the handpiece further comprises:
an opening disposed through the handpiece, wherein the opening permits a channel inside of the handpiece to be in fluid communication with the ambient atmosphere; and
the handpiece channel couples the opening of the handpiece to the channel of the working tip.

10. The ophthalmic surgical instrument of claim 9, wherein when the distal opening of the working tip is disposed inside the eye and the proximal opening is covered, covering the opening of the handpiece eliminates a pressure difference between the inside of the eye and inside of the handpiece channel.

11. The ophthalmic surgical instrument of claim 9, further comprising a valve mechanism configured to control flow of the fluid out of the opening of the handpiece and into the ambient atmosphere.

12. The ophthalmic surgical instrument of claim 9, wherein:
a tube connects the handpiece channel to a surgical console,
a distal end of the tube couples to an aperture of the ophthalmic surgical instrument, and
when the proximal opening of the working tip and the opening of the handpiece are covered, active aspiration provided by the surgical console is configured to aspirate fluid from inside the eye through the tube from the distal opening of the working tip.
